(19) United States
(12) Patent Application Publication
(10) Pub. No.: US 2003/0120307 A1
(43) Pub. Date: Jun. 26, 2003

(54) System and method for surgical enhancement of the lips

(76) Inventor: R. Patrick Abergel, Santa Monica, CA (US)

Correspondence Address: PERKINS COIE LLP, POST OFFICE BOX 1208, SEATTLE, WA, 98111-1208, US

(21) Appl. No.: 10/027,996

(22) Filed: Dec. 21, 2001

(51) Int. Cl. A61B 17/28

(52) U.S. Cl. 606/207; 128/898; 623/902

(57) ABSTRACT

In a surgical technique for augmentation of the lips, first and second openings are made on a lip. An implant inserting tool is inserted into the first opening and routed through the lip to and out of the second opening. The tool grabs or clamps onto an implant. The tool is then withdrawn back out through the first opening. The implant is released from the tool, with the implant remaining in the lip. The ends of the implant are trimmed and the openings closed with sutures. The surgical technique and tool minimize the risk of bruising or possible hematoma, reduces pain and trauma and allows for faster recovery. Similar techniques and tools may be used to treat skin depressions caused by acne scarring, and for treating nasal labial folds.

### BACKGROUND OF THE INVENTION

The human lips are an essential or even dominating, facial feature. Many, if not most facial expressions involve, or are characterized by, the form, position and shape of the lips. The lips contribute significantly to a person's appearance. Thin lips, especially in women, are considered unattractive in many cultures. With some individuals, the lips become thinner with age. With others, thinner lips are an inherent characteristic from birth. Regardless of the cause, many individuals have desired thicker or larger lips.

Various techniques have been proposed and used for lip enhancement. These, techniques include temporary injectable fillers, such as collagen. These fillers are injected into the lips to provide improved lip appearance, by enlarging or thickening the lips. However, since they are absorbed by the body over time, the result is only temporary, usually lasting from about one to six months. Other injection techniques include injection of a synthetic implant material, such as silicone. These materials provide a permanent effect. However, they are difficult or impossible to remove, and may involve other complications. Injectable bio-catalysts have also been used for lip augmentation. These types of products include synthetic materials, such as Gore-Tex PTFE, and bio-derived products, such as Alloderm, provide a permanent or semi-permanent result, often varying significantly between individuals. These type of bio-catalysts may generally be removed, if necessary, but with significant difficulty. Self-derivative implants have also similarly been used, such as fat, tendon or dermis grafting.

Apart from techniques using fillers or implants, other surgical techniques, such as lip-rolls, lip lifts, or micro pigmentation, have been used with varying degrees of success to provide the illusion of larger or fuller lips. Nonsurgical techniques, such as lip pumps, lip balms, or glosses, have also been used to try to achieve the same result of an improved appearance of the lips. However, these techniques have drawbacks, and uniformly provide only fleeting if any positive results.

Consequently, most patients prefer or require techniques which involve grafting or insertion of an implant. While use of implants generally provides very good results, disadvantages remain in the tools and procedures used to place the implant. Typically, a trocar or other relatively large tool having a cutting end is used to make the openings for insert placement. The trocar is then withdrawn and the implant placed into the opening made by the trocar, using a second tool, As a result, current implant placement procedures may involve significant risk of trauma, bruising or hematoma, pain, or risk of poor placement requiring removal.

Accordingly, it is an objection of the invention to provide improved methods and devices for implant lip augmentation surgery.

### SUMMARY OF THE INVENTION

To these ends, in a first aspect, a method for performing surgery on the lip of a human includes making first and second openings, incisions, or punctures at first and second locations on the upper or lower lip. Preferably, the locations or incision points are premarked. An insertion tool is inserted into the first opening and gently routed to the second opening. This creates a tunnel or opening through the lip tissue between the two openings. The leading end of the tool is moved out of the second opening. The implant is releasably attached to the leading end of the tool. Preferably, the leading end of the tool includes a clamp jaw, which may be clamped onto the implant. The tool is then pulled back towards the first opening, drawing the implant into the second opening and into the tunnel. The tool is then further withdrawn out from the first opening, along with the clamped or attached end of the implant. The implant is then released from the tool. The release step may be achieved by unlocking and/or opening the clamp jaws. The ends of the implant protruding out of the incisions are then preferably trimmed, and the incisions are closed. The foregoing method provides simple, rapid and reliable placement of an implant in the lip, with minimal risk of trauma, bruising or possible hematoma, as multiple needle punctures are avoided.

In a second aspect, a surgical tool is provided having jaws on its front or leading end. The jaws can be opened and closed via use of a lever or similar control at the handle. The front end of each of the upper and lower jaws is blunt. This allows the tool to create openings, pockets, or tunnels in tissue underlying the skin, without excessive cutting, bleeding, trauma or pain. A locking device may be provided to lock the jaws into an open or closed position. The tool is useful for performing various implant procedures.

In a third aspect, the tool is used to treat skin depressions resulting from acne scars or similar conditions. A pocket or recess is formed under the skin depression using the jaws to displace tissue. An insert is placed in the pocket. This lifts the skin, reducing or eliminating the skin depression. The need for multiple incisions is avoided.

In a fourth aspect, the tool is similarly used for treating nasal labial folds.

Other and further objects and advantages will appear hereinafter. The invention resides as well in subcombinations of the steps and elements described. The steps and elements of one embodiment may be used with, or in place of those of other embodiments described.

### BRIEF DESCRIPTION OF THE DRAWINGS

FIG. 1 is a prospective view of a lip implant inserter tool.

FIG. 2 is a side view thereof, with the jaws open.

FIG. 3 is a side view thereof, with the jaws closed.

FIG. 4 is an enlarged view of the jaws shown in FIG. 2.

FIG. 5 is an enlarged side view of the jaws shown in FIG. 3.

FIGS. 6A and 6B are enlarged perspective views thereof.

FIG. 7 is a diagram illustrating the techniques of the invention.

FIG. 8 is a perspective view of an implant kit containing an assortment of implants.

### DETAILED DESCRIPTION

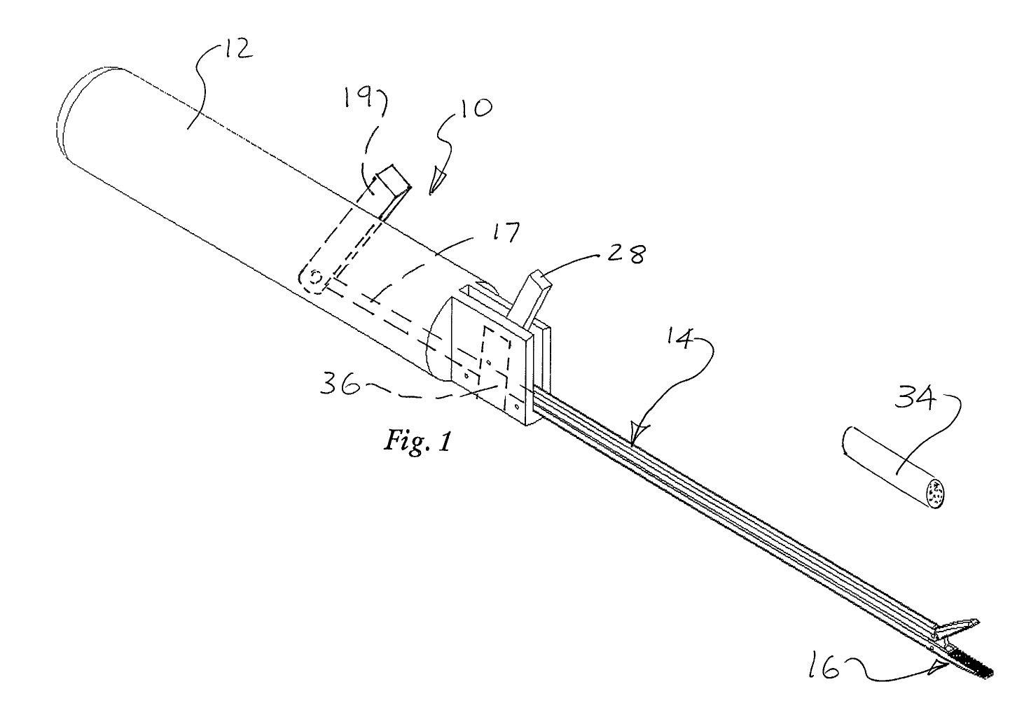

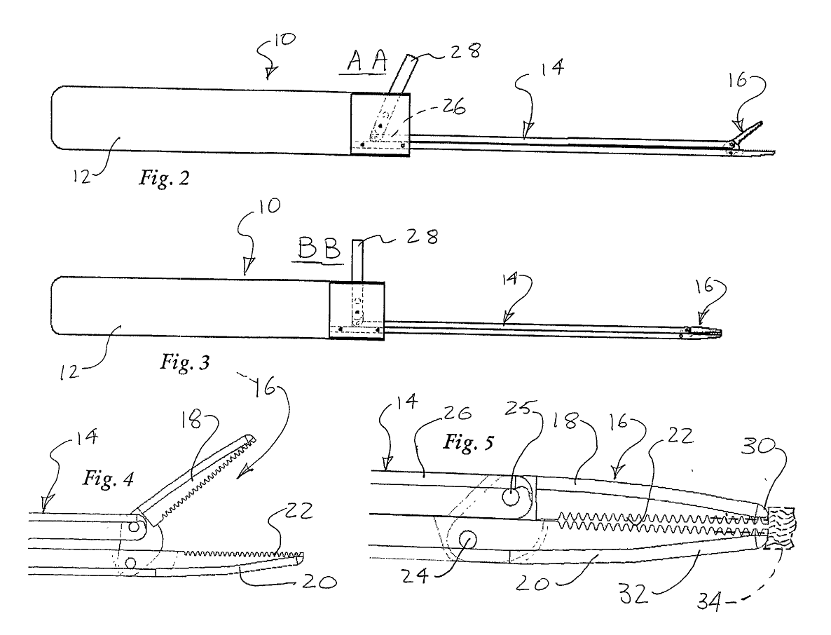

Turning now in detail to the drawings, as shown in FIGS. 1-6B, a lip implant inserter tool 10 has a body 14 attached to a handle 12. The handle and body are preferably cylindrical. A clamp jaw 16 is provided at the distal end of a body 14. The clamp jaw 16 preferably has an upper jaw 18 and a lower jaw 20, with each jaw including teeth or serrations 22. In the embodiment shown, the lower jaw 20 is fixed relative to the body 14 and handle 12. The upper jaw 18 pivots about a fixed pivot point 24 on the lower jaw 20 or the body 14, and about a moving pivot point 25 attached to a jaw linkage 26. Alternatively, the tool, can be designed so that both jaws can pivot outwardly. An actuating lever 28 on the handle 12 is attached to the back end of the linkage 26. Movement of the actuating lever 28 between the BB position shown in FIG. 3, to the AA position shown in FIG. 2, causes the upper jaw 18 to open and close. A detent or locking device 36 is preferably provided to lock the clamp jaw 16 into the closed position, as shown in FIGS. 3 and 5.

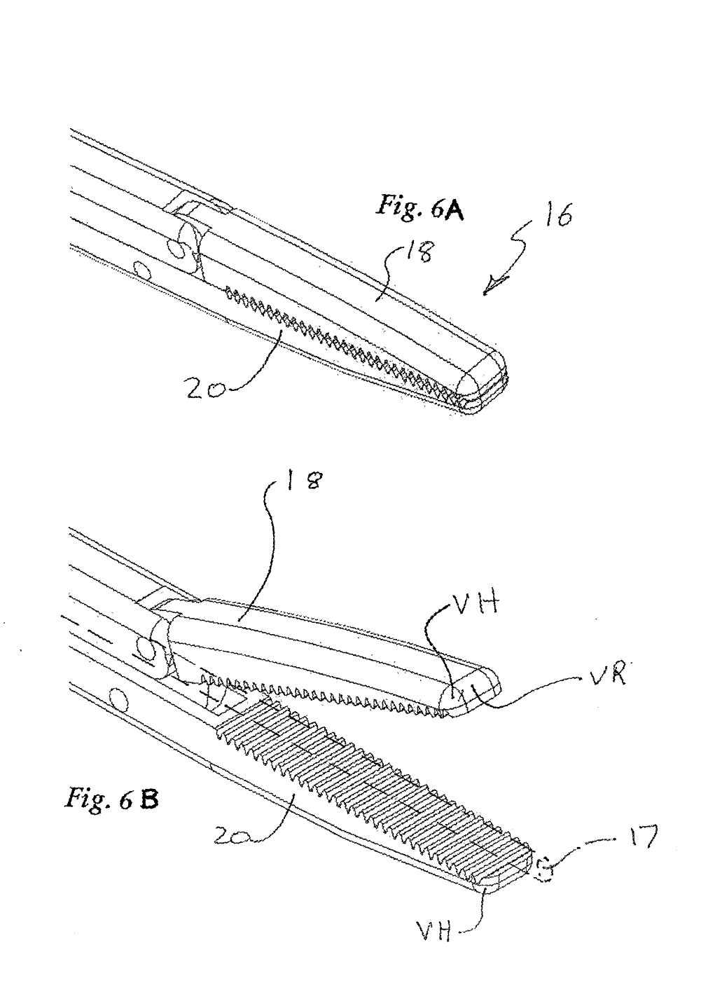

The leading ends of the jaws 18 and 20 are blunt. Referring to FIG. 6, the vertical radius VR and the horizontal radius VH at the tip of each jaw preferably ranges from 1 or 2 mm up to 4, 8, 6 or 10 mm.

The implant inserter tool 10 is used to insert an implant 34. The implant 34 may be a PTFE (polytetra fluoroethylene) lip implant, available from W. L. Gore & Associates. This material approved by the FDA for use in surgery and reconstruction of soft tissue. It has very low reactivity and is rarely associated with complications. It has been used for facial implantation for improvement of creases, folds and similar features. It is a permanent material and is not broken down by body. The implant may be a single strand or a multi-strand implant. Other implant forms and materials may also be used. Preferably, the implant is a biocompatible microporous material which supports rapid tissue incorporation and which remains flexible, soft, strong and permanent.

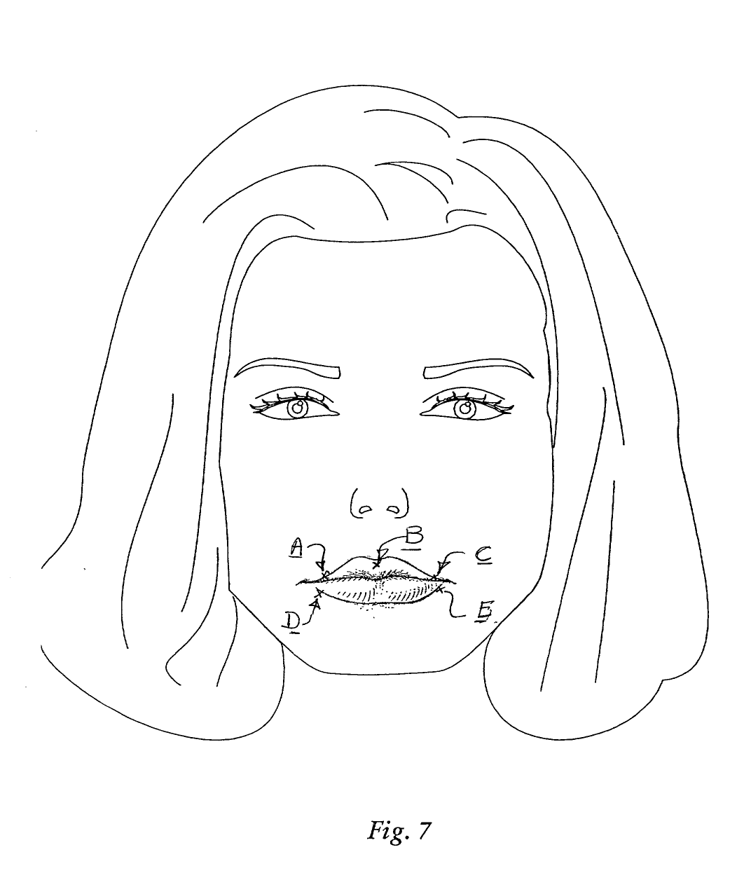

In use, after an informed consent has been completed, and pre-operative photographs have been taken, the vermillion border of the lip is outlined with a surgical marker. Referring to FIG. 7, for the upper lip, first and third incision points A and C are marked at the midline, and preferably about 3-10 mm, preferably 6 mm, inward from the corner of the mouth, on the mucosal part of the lip. The second central incision point B is preferably 1-4 mm and preferably 2 mm, below the vermillion border. For the lower lip, fourth and fifth incision points D and E are premarked 3-10 mm inward from the corner of the mouth (and preferably about 6 mm inward) on the mucosal part of the lip, and 1-4 mm and preferably 2 mm, below the vermillion border.

The patient is then placed on a surgical table. The perioral area is prepped in a sterile manner.

Preferably anesthesia is then provided. In the preferred method, 1/10th of a cc of lidocaine epinephrine mixture is used to anesthetize each of the five incision points. A 2-3 mm incision or puncture is made with a scalpel or other cutting instrument, at the previously marked incision points. An anesthetic solution is then infiltrated into each lip. Preferably, this is performed by infiltrating the lidocaine epinephrine solution using a blunt infiltrating needle (typically a 1 mm diameter, 4 inch length infiltrating needle).

With the inserter 10 in the closed and locked position, as shown in FIGS. 3, 5, and 6A, the leading end or clamp jaw 16 is inserted at the incision point A, as shown in FIG. 7. The leading end or clamp jaw 16 of the tool 10 is then gently guided from incision point A to incision point B, as shown in FIG. 7, following the outline of the vermillion boarder. The blunt nose 30 of the clamp jaw 16 displaces the lip tissue as the blunt nose 30 is moved from the incision point A through the lip tissue, to the incision point B. This creates an opening or tunnel through the lip tissue.

The clamp jaw 16 is moved out of the lip, through the incision point B. The clamp jaw 16 is then released or unlocked, by disengaging a detent or lock device 36. The actuator lever 28 is moved from position B in FIG. 3 to position AA in FIG. 2. This causes the clamp jaw 16 to open. In the specific design shown, this movement pulls back on the jaw linkage 26, causing the upper jaw 18 to pivot counterclockwise about the fixed pivot point 24, moving the clamp jaw 16 from the closed position shown in FIG. 5, to the open position shown in FIG. 4. One end of an implant 34 is then positioned between the upper jaw 18 and lower jaw 20. The actuating lever 28 is then moved back to the closed position BB, shown in FIG. 3. The locking device 36 preferably automatically reengages, locking the clamp jaw 16 in the closed position. The teeth or serrations 22 on the upper jaw 18 and lower jaw 20 securely grab the end of the implant 34, as shown in FIG. 5.

Following this technique minimizes the risk of bruising or possible hematoma, associated with multiple needle punctures.

The tool 10 is then gently withdrawn, pulling the implant 34 through the incision point B and into the lip. As the tool 10 is withdrawn, the implant 34 is pulled through the tunnel or opening in the lip tissue created by the initial forward movement of the blunt nose 30 from the incision point A to the incision point B. The tool 10 is withdrawn back out of the incision point A, along with the clamped or attached end of the implant. The locking device 36 is then disengaged, and the actuating lever 28 moved from position BB to position AA, as shown in FIGS. 3 and 2, causing the clamp jaw 16 to release from the implant 34. The implant 34 is now fully pulled or routed through the lip, with the ends of the implant protruding out of the incisions at points B and A. The two ends of the implant 34 are then preferably trimmed at a 45° angle, just below the skin surface.

The same procedure is then preformed on the other half of the upper lip using incision points B and C. On the lower lip, the same procedure is used via incision points D and E. No central incision point is generally needed or used on the lower lip, because the vermillion border on the lower lip is sufficiently straight to allow proper positioning of the implant directly between points D and E. After trimming the implant, incision points are closed with sutures (e.g. 6.0 polypropylene). Sutures are removed within 48-72 hours. With some patients, the incision point 5 at the lip centerline can be omitted and the tool 10 routed directly from point A to point C. Thus, the central incision point B may not be needed, depending on the shape of the lip and the nature of the underlying lip tissue.

The tool 10 may also be used to treat skin depressions caused by acne or other condition, while reducing the risk of bruising or possible hematoma associated with existing techniques relying on multiple needle punctures.

After an informed consent has been signed and pre-operative photographs have been taken, the skin depressions are outlined with a surgical marker. The patient is taken to the surgery room and the operative area is prepped in a sterile fashion. The patient is then placed on the surgical table and the perioral area is prepped in a sterile manner.

One-third of a cc of lidocaine epinephrine mixture is used to anesthetize each of the insertion points, one per cheek. The incision point is located at the distal end of the depression area. Then a preferably 2 to 3 millimeter puncture is made at an angle AN to the skin, preferably using a number 11 scalpel blade at the previously marked insertion point. The angle AN is preferably 20-70, 30-60, 40-50, or 45 degrees. The angled incision minimizes the risk of scarring. The lidocaine epinephrine solution is then injected subcutaneously, using a blunt-infiltrating needle, typically about 4 inches long.

Before the implant is inserted, the tool 10 is used to remove the adhesions causing for the skin depressions or scars. After inserting the tool 10 from the distal point, the jaws of the tool are positioned directly under the facial depression. Then, the adhesions are removed bluntly by a succession of opening and closing of the jaws, both on the horizontal and vertical planes. This creates a pocket or space under each depression. The tool 10 is then withdrawn from the surgical site.

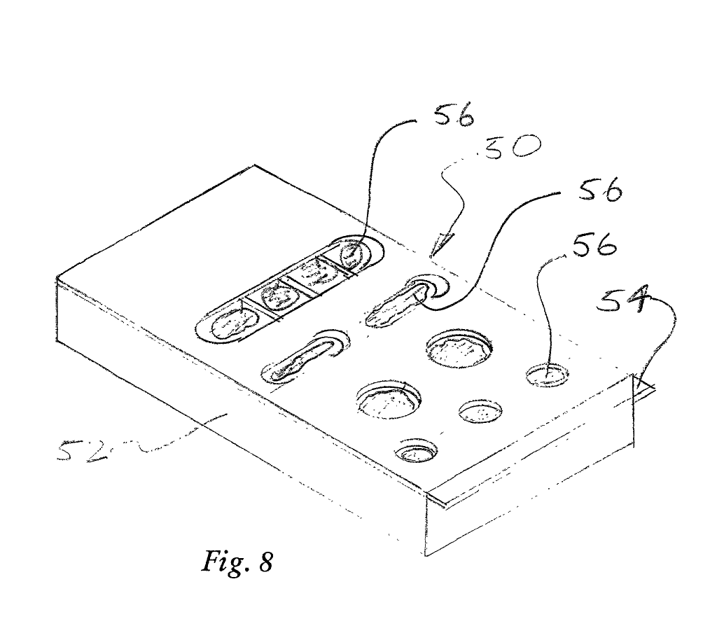

The surgeon selects a pre-cut implant, preferably from an array of pre-cut implants of varying size. FIG. 8 shows an implant kit 50 containing pre-cut implants. The implant kit 50 includes a base or tray 52 having multiple compartments. Each compartment holds an implant 56. The compartments and the implants contained in them are of varying shape and size. The compartments are covered and sealed via a peel off lid 54. The lid is peeled off or opened during or immediately before the surgical procedure, to maintain sterility. The surgeon selects an implant from the kit, based on the size and shape of implant needed for the procedure.

The selected implant is held or preferably clamped in the jaws of the tool 10. The tool is manipulated to place the insert into the pocket previously created. After the insert is properly positioned, the jaws of the tool are released and opened up. An ejector 27 at the jaws is extended to push the insert out of the jaws. The ejector is controlled by a second lever 19, or by a third position of the lever 28 (e.g., moving the lever 28 beyond the open position) shown in FIGS. 1 or 6. The insert and the tool is withdrawn, leaving the insert in place in the pocket. The incision is closed using a suture. After closure, a steri-strip is applied over the suture. The procedure may be repeated at another site, with another implant selected from the implant kit. The suture is removed after 48 hours.

Nasal labial folds may also be treated using the tool and techniques described above. For treatment of nasal labial folds, the nasal labial folds are outlined with a surgical marker. One-tenth of a cc of lidocaine epinephrine mixture or other anesthesia solution is injected intradermally at each extremity of the nasal labial fold. A first e.g., three-millimeter incision is made, preferably using a scalpel blade, at a first end, and a second incision or puncture is made at the second end of the nasal labial fold. Two cc of the anesthetic solution is then injected subcutaneously into each nasal labial fold using a blunt infiltrating needle.

With the jaws of the tool 10 in a closed and locked position, the tool is inserted through the first incision and is moved through the tissue to the second (typically the lower) end of the nasal labial fold. This creates a tunnel path in the tissue. After moving out of the second incision, the jaws are unlocked and opened, and the implant is positioned in and clasped by the jaws. The jaws are then locked in the closed position. The surgeon then gently pulls the tool back. The tool automatically guides the implant through the precise tunnel path it created on the way in. The implant is released by unlocking and opening the jaws. Each of the two edges of the implant is trimmed at a 45-degree angle. The same procedure is then performed on the other nasal labial fold. The insertion points are closed using a suture. After closure, a steri-strip is applied over the suture. Sutures are removed after 48 hours.

Various equivalents and substitutions may of course be made. For example, the temporary engagement, attachment or clamping provided by the clamp jaw may alternatively be provided by equivalents, such as hooks, loops, friction attachments, temporary adhesives, or other equivalent ways to temporarily attach the implant 34 to the tool 10. The location of the incision points may of course also be moved, as may be required for the characteristics of the patient or the desired result to be achieved. Many patients have some irregularity or unevenness in the size and shape of the lips e.g., due to accidents, trauma, etc. The foregoing methods can also be used to minimize or correct these conditions. The invention, therefore, should not be restricted, except to the following claims, and their equivalents.

1. A method for performing surgery on a lip of a human, comprising the steps of:
making a first opening at a first location on the lip;
making a second opening at a second location on the lip;
inserting a tool into the first opening;
routing the tool through the lip to the second opening;
moving the a leading end of the tool at least partially out of the second opening;
attaching the leading end of the tool to an implant;
pulling the leading end of the tool and the implant back into the lip towards the first opening;
separating the implant from the leading end of the tool, with the implant positioned within the lip; and
withdrawing the leading end of the tool out of the first opening:

2. The method of claim 1 further comprising the step trimming the ends of the implant.

3. The method of claim 1 wherein the leading end of the tool includes a clamp jaw, and the leading end of the tool is attached to the implant by closing the clamp jaw onto an end of the implant.

4. The method of claim 3 wherein the implant is separated from the leading end of the tool by at least partially opening the clamp jaw.

5. The method of claim 3 further comprising the step of locking the clamp jaw closed onto the implant.

6. The method of claim 1 wherein the first opening and the second opening are made by puncturing the lip.

7. The method of claim 1 further comprising the step of pre-marking the first and second openings.

8. The method of claim 1 wherein the lip is the upper lip of a human patient.

9. The method of claim 1 wherein the first opening is 3-10 mm inward from the corner of the mouth, on the mucosal part of the lip.

10. The method of claim 7 wherein the first opening is 1-4 mm below the vermillion border of the lip.

11. The method of claim 1 wherein the routing step forms a tunnel path through the lip from the first opening to the second opening.

12. The method of claim 1 further comprising the step of infiltrating an anesthetic solution into at least one of the first and second openings.

13. The method of claim 1 wherein the routing step is performed by guiding the leading end of the tool to follow the vermillion border.

14. A tool for surgically inserting an implant into a lip of a human, comprising:
a handle;
an elongate body having a first end and a second end, with the first end attached to the handle;
a clamp jaw on the second end of the elongate body;
a clamp jaw operating element on the handle and linked to the clamp jaw, for opening and closing the clamp jaw.

15. The tool of claim 14 with the elongate body having a diameter of 1-6 mm.

16. The tool of claim 14 further comprising a jaw lock linked to the clamp jaw, for holding the clamp jaw in a closed position while the jaw lock is actuated.

17. The tool of claim 14 further comprising a lip implant attachable to the tool via the clamp jaw clamping onto the lip implant.

18. The tool of claim 14 with the clamp jaws each having a blunt end nose tapering outwardly to a diameter equal to or greater than the diameter of the elongate body.

19. The tool of claim 18 with the blunt end nose having a diameter of 1-6 mm.

20. A method for treating skin depressions comprising the steps of:
making an incision adjacent to a first end of the skin depression;
inserting a tool into the incision;
creating a pocket under the skin depression via manipulation of the tool;
withdrawing the tool from the incision;
securing an insert onto the tool;
moving the tool and the insert through the incision and positioning the insert in the pocket; and
withdrawing the tool from the incision.

21. The method of claim 20 wherein the tool includes jaws and wherein the pocket is created by opening and closing the jaws while the jaws are under the skin depression.

22. The method of claim 21 wherein the insert is secured onto the tool by clamping at least part of the insert between the jaws.

23. The method of claim 20 further comprising the step of injecting an anathetic solution subcutaneously by placing a blunt infiltrating needle into the incision.

24. The method of claim 20 wherein the incision is made at a angle to the skin surface ranging from 30-60 degrees.

25. A method for treating nasal labial folds, comprising the steps of:
making a first incision adjacent to a first end of the nasal labial fold;
making a second incision adjacent to a second end of the nasal labial fold;
inserting a tool through the nasal labial fold by inserting a leading end of the tool into the first incision and routing it to and out of the second incision;
attaching an implant to the leading end of the tool;
pulling the tool and part of the implant back out of the first incision;
releasing the tool from the implant.

26. The method of claim 25 further comprising the step of trimming the ends of the implant.

27. The method of claim 25 wherein the leading end of the tool includes jaws and the wherein the implant is attached to the tool by clamping a part of the implant between the jaws.

28. The method of claim 27 wherein the jaws have a blunt front end and the routing step creates a tunnel via movement of the blunt front end from the first incision to the second incision.

29. The tool of claim 14 wherein the blunt end nose of each clamp jaw has a radius of from 1-6 mm.

30. The tool of claim 14 with the clamp jaw having an upper and a lower jaw, and further comprising an ejector for pushing an insert out from between the upper and lower jaw.
